(19) United States
(12) Patent Application Publication
(10) Pub. No.: US 2011/0197312 A1
(43) Pub. Date: Aug. 11, 2011

(54) Indigenous Viral Satellite RNA Associate with Cucumber Mosaic Virus used as a Biological Control Agent against Tomato Diseases caused by Virus Strains, Viroids, and Fungi

(75) Inventor: Magdy Shaban Montasser, Safat (KW)

(73) Assignee: UNIVERSITY OF KUWAIT, Safat (KW)

(21) Appl. No.: 12/701,582

(22) Filed: Feb. 7, 2010

(51) Int. Cl. A01H 1/00; C12N 15/63

(52) U.S. Cl. 800/279; 435/320.1

(57) ABSTRACT

Viral satellite RNA associated with cucumber mosaic virus KU1 strain used as a biological control agent against a severe viral strain KU2 caused tomato necrosis. Potato spindle tuber viroid (PSTV) caused tomato stunting, fusarium wilt disease in tomato caused by Fusarium oxysporum f. sp. Lycopersicae and leaf spotting disease in tomato caused by Alternaria alternata were also put under control using the same biological control agent. The virus strains, viroids and fungi caused tomato diseases were biologically controlled at the molecular level using a molecular parasite occurred naturally associated with a viral genome of an indigenous strain of cucumber mosaic virus.

### BACKGROUND OF INVENTION

1. Field of invention

The present invention relates to Indigenous Viral Satellite RNA Associate with Cucumber Mosaic Virus and in more particular one the works as a Biological Control Agent against Some Tomato Diseases.

2. Description of Prior Art

Biological control technology based on viral satellite mediated protection is a novel concept of the viral disease control strategies. Plant virus diseases cause severe constraints on the productivity of wide range economically important crops worldwide. Among these plant viruses, Cucumber mosaic virus (CMV) is considered one of the most economically damaging viruses among field grown vegetables. This virus infects over 1000 species of host plants belonging to 85 plant families, making it the broadest host range virus known. Outbreaks of diseases incited by CMV have caused large losses of tomato and pepper crops in several Mediterranean. In the United States, high incidences of CMV have been reported in several states.

CMV can harbor molecular parasites known as satellite RNAs that dramatically alter the symptom phenotype induced by the virus. Over 40 CMV satellite RNA isolates have been identified and sequenced, most reduce the viral disease to a virtually asymptomatic condition, and while relatively few increase the severity of virus symptoms. Thus, satellites have been referred to as “natural inhibitors of crop damaging viruses’.

These satellites RNAs are small nucleic acids whose nucleotide sequences are unrelated to, but are replicatively dependent upon the viral genome; they have a molecular parasitic relationship. According to research, the satellite RNA of CMV which was designated as CMV-associated RNA 5 (CARNA 5) or satRNA modulated the symptom expression of its helper CMV. The satellite parasitizes the virus at molecular level by competing with latter for the host plant's enzymatic machinery and replicative enzymes that are essential in virus multiplication and infection. The satellite's competition literally causes the virus to starve and serves us the conceptual basis for experiments on biological control of CMV in the field conducted in China. The attenuating properties of some satellite RNAs can be exploited to control viral diseases by preinoculating crops with an attenuating satellite RNA variant combined with virus. This technique can effectively protect against CMV disease in the field.

### PRIOR ART

U.S. Pat. No. 7,294,758 by Polston, et al. and issued on Nov. 13, 2007 is for materials and methods for producing geminivirus resistant plants. It discloses materials and methods for producing plants that are resistant to infection by geminiviruses and other related viruses. The invention comprises transforming a plant with a polynucleotide wherein when the polynucleotide is expressed in the plant, the transformed plant exhibits resistance to plant viral infections. Exemplified herein is the use of a polynucleotide encoding a Rep protein derived from tomato mottle geminivirus.

U.S. Pat. No. 6,818,804 by Gonsalves, et al. and issued on Nov. 16, 2004 is for Tospovirus resistance in plants. It discloses nucleotide sequences for tomato spotted wilt virus (TSWV) nucleocapsid and transgenic plants containing the nucleocapsid nucleotide sequence from a TSMV isolate is shown to provide resistance in the transgenic plant to Tospoviruses from different serogroups. In addition, transgenic plants containing the nucleocapsid nucleotide sequence from a lettuce isolate of TSWV were produced and shown to provide (in plants producing small amounts of the nucleocapsid protein) resistance in the transgenic plant to both homologous and closely related viral isolates whereas plants producing larger amounts of the nucleocapsid protein possessed moderate levels of protection against both the homologous isolate and isolates of distantly related Impatiens necrotic spot virus (INSV).

U.S. Pat. No. 6,207,882 by Ding and issued on Mar. 27, 2001 is for a disease resistant transgenic plants comprising a tomato aspermy virus 2b gene. It discloses transgenic plants transformed with a tomato aspermy virus 2b gene or active fragment thereof exhibit resistance to diseases caused by infectious pathogens, such as viruses. Expression of the 2b gene causes activation of hypersensitive response and expression of pathogenicity-related proteins in plants that are incapable of such a response to certain pathogens. Transformation of a wide variety of plants with expression vectors in which the tomato aspermy virus 2b gene is operably linked to a plant-active promoter renders the plant resistant to pathogenic infection.

United States Patent Application 20050289671 by Polston, et al. and published on Dec. 29, 2005 is for materials and methods for producing geminivirus resistant plants. It discloses materials and methods for producing plants that are resistant to infection by geminiviruses and other related viruses. Methods of the invention comprise transforming a plant with a polynucleotide wherein when the polynucleotide is expressed in the plant, the transformed plant exhibits resistance to plant viral infections. Exemplified herein is the use of a polynucleotide encoding a Rep protein derived from tomato mottle geminivirus. The methods of the invention can be used to provide resistance to viral infection in plants such as tomato and tobacco. The invention also concerns transformed and transgenic plants in plant tissue that express a polynucleotide encoding a plant virus Rep protein, or a fragment or variant thereof.

There is still room for improvement in the art.

### SUMMARY OF INVENTION

The current invention involves the using of viral satellite RNA associated with cucumber mosaic virus KU1 (Kuwaiti Strain #1) strain as a biological control agent against a severe viral strain KU2 (Kuwaiti Strain #2) caused tomato necrosis. Potato spindle tuber viroid (PSTV) caused tomato stunting, fusarium wilt disease in tomato caused by Fusarium oxysporum f. sp. Lycopersicae and leaf spotting disease in tomato caused by Alternaria alternata were also put under control using the same biological control agent.

The virus strains, viroids and fungi caused tomato diseases were biologically controlled at the molecular level using a molecular parasite occurred naturally associated with a viral genome of an indigenous strain of cucumber mosaic virus.

This is more efficient, effective, accurate and functional than the current art.

### BRIEF DESCRIPTION OF DRAWINGS

Without restricting the full scope of this invention, the preferred form of this invention is illustrated in the following drawings:

There are no Current Drawings with this application.

### DETAILED DESCRIPTION

The following description is demonstrative in nature and is not intended to limit the scope of the invention or its application of uses.

There are a number of significant design features and improvements incorporated within the invention.

The current invention involves the using of viral satellite RNA associated with cucumber mosaic virus (CMV) KU1 strain as a biological control agent against a severe viral strain KU2 caused tomato necrosis. Potato spindle tuber viroid (PSTV) caused tomato stunting, fusarium wilt disease in tomato caused by Fusarium oxysporum f. sp. Lycopersicae and leaf spotting disease in tomato caused by Alternaria alternata were also put under control using the same biological control agent.

A naturally occurring satellite RNA parasite the viral genome at the molecular level was discovered in KU1 strain. This viral parasite was first characterized, and then used as a biological control agent to protect tomato plants against not only the disease induced by severe CMV straints KU2, but also against potato spindle tuber viroids (PSTV), and fungal diseases caused by Fusarium sp and Alternaria sp. in tomato. In plant growth chambers, tomato plants were pre inoculated or “vaccinated” with KU1 containing viral satellite RNA and then challenge inoculated with a severe strain at different time intervals. All plants challenged three weeks after vaccination (or pre-inoculation) showed nearly complete protection from subsequent infection by severe strains. This biological control technology of plant viruses was found protective and could be successfully established sooner after vaccination.

Two strains of cucumber mosaic virus (CMV) isolated in Kuwait confirmed their infectivity based on symptomatology and host range on different cultivars of tomato (Lycopersicon esculent), tobacco (Nicotiana tabacum L.) and squash (Cucurbita pep) that were found susceptible to both strains of cucumber mosaic virus. The pattern of symptoms appearance differed for the two strains in tomato and tobacco, showing severe symptoms with one strain designated KU3 and almost symptom less with the other strain designated KU1 strain of the virus. White Bush F1 hybrid cultivar of squash which was found highly susceptible to both viral strains in comparison to other cultivars showed an exceptional feature of local symptoms appearance along with known systemic symptoms. Viability and infectivity of these strains in extracted nucleic acid inoculums was further proved by mechanical transmission. Increased nucleic acid yields as double stranded DNA, RNA and single stranded RNA were also notice in virus infected plants compared to healthy plants with less yield. A naturally occurring satellite RNA parasite the viral genome at the molecular level was discovered in KU1 strain. This viral parasite was first characterized, and then used as a biological control agent to protect tomato plants against not only the disease induced by severe CMV strains, but also against potato spindle tuber viroids (PSTV), and a fungal diseases caused by Fusarium oxysporum f. sp. Lycopersicae and Alternaria alternata in tomato.

The symptomatology was checked by inoculating both KU1 and 2 CMV strains on test plants by mechanical sap transmission. Inocula were prepared by grinding CMV infected tissues (1g/9 ml) in 0.01 M potassium phosphate buffer (pH 7.1). The leaves of the young test plants were dusted with 600-mesh carborundum and then with a cotton swab, ground CMV infected tissues were rubbed over those leaves. Tomato plants showed severe infections when inoculated with TNA inoculums of KU2 infected tissues, but were symptom less in the inoculation with TNA from KU1 infected plants. Chlorotic spots and local chlorotic lesions were observed on squash cv. White Bush F1 hybrid inoculated with TNAs from either KU1 or KU2 infected plants.

Gel electrophoretic analyses show an average molecular weights of 3.3 kb and 1.0 kb were noticed in KU1 and KU2 infected squash TNA extracts, by analyzing on a 6% gel at 200 V, while no band at that distance in the healthy were observed. Nucleic acid bands of 374 by and 400 by appeared in KU1 infected plants. Similarly, KU1, KU1 challenged with KU2 extracts and KU2 alone infected tomato extracts showed a 3.5 kb band when they were analyzed on a 6% gel at 300 V. There were some extra bands of 292 by and 330 by in KU1 and KU1 challenged with KU2 extracts but these bands were missing in healthy and KU2 alone infected extracts.

CMV is a wide host range virus, differing in symptom appearance pattern according to the viral strain used. Test plants selected for experiment were susceptible to viral infection by both strains KU1 and KU2 of CMV. Results showed that the strains differed from each other, in the way of appearance of symptoms on different hosts and depending upon that were considered as mild strain KU1 and severe strain KU2 of CMV. The severe KU2 strain showed stunting, chlorosis and mosaic symptoms on tomato and sever mosaics on squash as well as on tobacco leaves. However, KU1 strain was found as a mild strain showing mosaic on squash leaves but symptom less on tomato. Similarly, in tobacco plants also, this KU1 strain showed mild mosaics on very young leaves but later the plants were found symptom less. One peculiar feature observed for the first time during this investigation was the appearance of local chlorotic spots on the cotyledon leaves of cultivar White Bush F1 hybrid squash along with the systemic symptoms on the rest of plant leaves. These local symptoms were more severe in plants inoculated with the KU2 strain. Extracted CMV nucleic acid was also viable and effective in inoculation test. This was proved by back inoculating the test plants with extracted nucleic acids of virus-infected plants.

Preliminary level preinoculation and challenge inoculation showed a complete protection in plants challenged 3 weeks after preinoculation, followed by 2 weeks and least prevention in plants challenged 1 week after preinoculatinon. The results demonstrate that introduction of a non-estrogenic CMV satellite via preinoculation or “vaccination”, into tomato plants will prevent severe disease following later infection by a severe strain of CMV. The preinoculation technique has the advantage of providing a rapid response to viral epidemics and the satellite mediated protection of tomato and pepper against CMV infection has been tested.

The invention works against fungal diseases as well as PSTV

### ADVANTAGES

The current invention is the development of a method for better and a suitable “vaccine” production by using viral satellite of mild strain as biological control agent to protect economically important tomato crops against other severe viral diseases. Thus the demonstration of the effectiveness of preinoculation or “vaccinatinon” against a natural challenge infection emphasizes the usefulness of the method of satellite mediated biological control of CMV.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided. With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A Biological Control Agent comprising:
a viral satellite RNA associated with the helper viral genome being used as a biological control agent against severe strains of CMV.

2. The agent as described in claim 1, further comprising being isolated from Kuwait environment as an indigenous strain.

3. The agent as described in claim 1, further comprising being used to control fungal diseases affecting tomato crops caused by Fusarium oxysporum f. sp., Lycopersicae and Alternaria alternata.

4. The agent as described in claim 1, further comprising being used to control potato spindle tuber viroids (PSTV) in tomato crops.

5. The agent as described in claim 1, further comprising using said agent to pre-inoculated plants.

6. The agent as described in claim 5, further comprising where said plants are tomato plants.

7. A method comprising:
using a viral satellite RNA associated with the helper viral genome as a biological control agent against severe strains of CMV.

8. The method as described in claim 7, further comprising being isolated from Kuwait environment as an indigenous strain.

9. The method as described in claim 7, further comprising being used to control fungal diseases affecting tomato crops caused by Fusarium oxysporum f. sp., Lycopersicae and Alternaria alternata.

10. The method as described in claim 7, further comprising being used to control potato spindle tuber viroids (PSTV) in tomato crops.

11. The method as described in claim 7, further comprising using said agent to pre-inoculated plants.

12. The method as described in claim 12, further comprising where said plants are tomato plants.
